(19) United States
(12) Patent Application Publication
(10) Pub. No.: US 2020/0368326 A1
(43) Pub. Date: Nov. 26, 2020

(54) USE OF IGF-1 IN THE PREPARATION OF MEDICAMENTS FOR PREVENTING AND/OR TREATING LIVER TUMORS

(71) Applicant: Dalian Medical University, Dalian (CN)

(72) Inventors: Aiguo WANG, Dalian (CN); Jingyu WANG, Dalian (CN); Xu ZHENG, Dalian (CN); Xiaoqin LUO, Dalian (CN); Huiling LI, Dalian (CN)

(21) Appl. No.: 16/877,907

(22) Filed: May 19, 2020

(30) Foreign Application Priority Data
May 20, 2019 (CN) 201910418231. 7

(51) Int. Cl. A61K 38/30; A61P 35/00

(52) U.S. Cl. CPC A61K 38/30; A61K 9/0019; A61P 35/00

(57) ABSTRACT

The present invention provides a use of IGF-1 in the preparation of medicaments for preventing and/or treating liver tumors, relating to the technological field of prevention and treatment of hepatocarcinoma. In embodiments of the present invention, experiments are carried out on mice, demonstrating that over-expression of IGF-1 in hepatocytes induces the apoptosis of cancerous cells indirectly, and inhibits the occurrence and development of liver tumors; high level of IGF-1 significantly promotes the function of thymus, delays the degeneration of thymus, and does not cause the dysfunction of thymus; high level of IGF-1 significantly promotes the function of spleen T lymphocyte; high level of IGF-1 significantly enhances the number and activity of tumor infiltrating T lymphocytes; long-term over-expression of IGF-1 does not significantly promote the incidence of tumors in all organs and tissues in mice, indicating the safety of high IGF-1 level in the body.

### TECHNICAL FIELD

The present invention pertains to the technological field of prevention and treatment of hepatocarcinoma, and specifically pertains to a use of IGF-1 in the preparation of medicaments for preventing and/or treating liver tumors. Insulin-like Growth Factor (IGF).

Further, this patent application incorporates by reference the Sequence Listing file enclosed herewith having the file name “SEQ.LISTING.txt” which is comprised of 1,185 bytes and has a date of creation of Apr. 27, 2020.

### BACKGROUND

Primary hepatocellular carcinoma (HCC) accounts for over 90% of primary hepatocarcinoma (hereinafter referred to as hepatocarcinoma), and is one of the three most fatal cancers in China. Patients with hepatocarcinoma often have no early warning signs or symptoms, develop rapidly and are highly malignant, and usually live an average of three to six months after diagnosis.

The liver is not only the largest digestive and metabolic organ of human, also an important immune organ. Gastrointestinal blood entering the liver through the portal venous system is rich in a large amount of potential antigens (including microorganism that normally inhabits in the gastrointestinal system, digested food, and pathogens in the infectious state), requiring the immunologic tolerance of liver to vast majority of harmless antigens. Therefore, liver is a special immune privilege organ in the body, in which the immune system is in suppression, this is why liver transplantation is easy to succeed.

However, at the same time, liver would promptly initiate specific and non-specific immune responses to harmful antigens such as lipopolysaccharide, bacterial superantigens and the like, and cause inflammations. In liver damage induced by virus infection, excessive alcohol intake and nonalcoholic steatohepatitis, persistent inflammation not only leads to liver fibrosis, liver cirrhosis and hepatocarcinoma, it is also accompanied by the imbalance of immune microenvironment in the liver, which is also the characteristic feature in patients with hepatocarcinoma. Moreover, hepatocarcinoma cells themselves also have the ability to regulate the immune system, further promoting the occurrence and development of cancers.

Many advances have been made in immuno-biological therapy for hepatocarcinoma in recent years, while there are few related clinical studies on immuno-biological therapy so far and the therapeutic effects are poor, the strong immunosuppressive state of liver and liver tumor microenvironment is one of the most important reasons. The modulation strategy of immune suppression in microenvironment and inflammatory immune imbalance in hepatocellular carcinoma will bring new hope for the immuno-biological therapy of hepatocellular carcinoma. However, no effective immune modulation factor has been found.

### SUMMARY

In view of this, the present invention aims to provide a use of IGF-1 in the preparation of medicaments for preventing and/or treating liver tumors. Increasing the level of IGF-1 in the body can effectively enhance the function of thymus, increase the production of T cells, improve the proliferation capacity and activity of peripheral T cells, enhance the quantity and killing power of tumor infiltrating T cells, and further inhibit the occurrence and development of liver tumors.

To achieve the above objectives, the present invention provides the following technical solution:

The present invention provides a use of IGF-1 in the preparation of medicaments for preventing and/or treating liver tumors.

The present invention provides a use of IGF-1 in the preparation of medicaments for preventing and/or treating liver tumors by promoting the function of thymus.

The present invention provides a use of IGF-1 in the preparation of medicaments for preventing and/or treating liver tumors by promoting the function of spleen T lymphocytes.

The present invention provides a medicament for preventing and/or treating liver tumors, comprising IGF-1 and excipients.

The present invention provides a use of IGF-1 in the preparation of medicaments for preventing and/or treating liver tumors. In embodiments of the present invention, experiments are carried out on mice, demonstrating that over-expression of IGF-1 in hepatocytes induces the apoptosis of cancerous cells indirectly, and inhibits the occurrence and development of liver tumors; high level of IGF-1 significantly promotes the function of thymus, delays the degeneration of thymus, and does not cause the dysfunction of thymus; high level of IGF-1 significantly promotes the function of spleen T lymphocytes; high level of IGF-1 significantly enhances the number and activity of tumor infiltrating T lymphocytes; long-term over-expression of IGF-1 does not significantly promote the incidence of tumors in all organs and tissues in mice, indicating the safety of high IGF-1 level in the body.

### BRIEF DESCRIPTION OF THE DRAWINGS

FIG. 1 shows mRNA expression level of Hras12V oncogenes in Hras12V^{+/+} and Hras12V^{+/+}/IGF1⁺ model mice;

FIG. 2 shows the level of IGF-1 in the serum of Hras12V^{+/+} and Hras12V^{+/+}/IGF1⁺ model mice;

FIG. 3 is a general anatomical drawing of the liver of Hras12V^{+/+} and Hras12V^{+/+}/IGF1⁺ model mice;

FIG. 4 is a diagram showing the liver/body weight ratio results of Hras12V^{+/+} and Hras12V^{+/+}/IGF1⁺ model mice;

FIG. 5 is a diagram showing the size and number of liver tumor in Hras12V^{+/+} and Hras12V^{+/+}/IGF1⁺ model mice;

FIG. 6 is a diagram comparing the contents of IGF-1 in the serum of IGF1⁺ and wild type C57BL/6J mice;

FIG. 7 is a general anatomical drawing showing the occurrence and development of liver tumor in C57BL/6J and IGF1⁺ mice co-induced with DEN, CCL4, and alcoholic carcinogens;

FIG. 8 is a diagram comparing the size and number of tumor in the liver of C57BL/6J and IGF1⁺ mice co-induced with DEN, CCL4, and alcoholic carcinogens;

FIG. 9 is a diagram showing the liver/body weight ratio results of C57BL/6J and IGF1⁺ mice co-induced with DEN, CCL4, and alcoholic carcinogens;

FIG. 10 shows the effect of IGF-1 on the proliferation of Hep3B, HepG2, Hep1-6 hepatocarcinoma cells cultured in vitro;

FIG. 11 shows the effect of IGF-1 on the apoptosis of Hep3B, HepG2, Hep1-6 hepatocarcinoma cells cultured in vitro;

FIG. 12 shows the effect of Hras12V^{+/+} mice and Hras12V^{+/+} IGF1⁺ mice on signaling pathways of liver tumor tissues and tumor-surrounding tissues;

FIG. 13 shows the effect of Hras12V^{+/+} IGF1⁺ mice on apoptosis pathways Caspase 3 and Caspase 8 of liver tumor and liver tumor-surrounding tissues;

FIG. 14 shows the effect of high level of IGF-1 on the thymus weight and index of mice;

FIG. 15 shows the effect of high level of IGF-1 on the number of cells in the thymus of mice;

FIG. 16 shows the effect of high level of IGF-1 on the function of thymus of mice;

FIG. 17 shows the effect of high level of IGF-1 on the spleen weight and index of mice;

FIG. 18 shows the effect of high level of IGF-1 on the number of cells in the spleen of mice;

FIG. 19 shows the effect of high level of IGF-1 on the function of spleen of mice;

FIG. 20 shows the effect of high level of IGF-1 on the activities of spleen CD4 and CD8 cells of mice;

FIG. 21 shows the effect of high level of IGF-1 on the proliferation capacity of spleen T lymphocyte;

FIG. 22 shows the effect of high level of IGF-1 on the number of tumor infiltrating lymphocytes;

FIG. 23 shows the effect of high level of IGF-1 on the proportion of CD8/CD4 cells in the tumor infiltrating lymphocytes;

FIG. 24 shows the effect of high level of IGF-1 on the activities of tumor infiltrating T lymphocytes;

FIG. 25 is a general anatomical drawing showing the effect of exogenous IGF-1 on the occurrence and development of liver tumor induced with Ras oncogenes;

FIG. 26 shows the effect of exogenous IGF-1 on the liver and body weight during the occurrence and development of liver tumor induced with Ras oncogenes;

FIG. 27 shows the effect of exogenous IGF-1 on the size and number of tumors during the occurrence and development of liver tumor induced with Ras oncogenes.

### DESCRIPTION OF THE EMBODIMENTS

The present invention provides a use of IGF-1 in the preparation of medicaments for preventing and/or treating liver tumors.

The IGF-1 of the present invention is preferably generated by over-expression of the body itself. There is no special limitation on the over-expression processes in the present invention, and any conventional over-expression processes in the art can be used. There is no special limitation on the dosage forms of the medicaments in the present invention, and any conventional dosage forms in the art can be used.

The present invention provides a use of IGF-1 in the preparation of medicaments for preventing and/or treating liver tumors by promoting the function of thymus.

The present invention provides a use of IGF-1 in the preparation of medicaments for preventing and/or treating liver tumors by promoting the function of spleen T lymphocytes.

The present invention provides a medicament for preventing and/or treating liver tumors, comprising IGF-1 and excipients.

The use of IGF-1 in the preparation of medicaments for preventing and/or treating liver tumors provided in the present invention will be illustrated in detail below in combination with embodiments, which could not be construed as limiting the protection scope of the invention.

### Embodiment 1

Over-Expression of IGF-1 in Hepatocytes can Significantly Inhibit the Occurrence and Development of Liver Tumor Induced with Ras Oncogenes

I. Preparation of Model Mice

(1) Hras12V^{+/+} transgenic mice with hepatocarcinoma: transgenic mice with Hras12V oncogenes specifically expressed in the hepatocytes (Journal of Hepatology, 2005, 43(5):836-844).

(2) IGF1⁺ transgenic mice: transgenic mice with IGF-1 specifically over-expressed in liver (Endocrinology, 2009, 150(9):4395-4403).

(3) Hras12V^{+/+}/IGF1⁺ model mice: Hras12V transgenic mice are hybridized with IGF1 mice to screen Hras12V^{+/+}/IGF-1±mice. Hras12V and IGF-1 genes are both over-expressed in hepatocytes of such model mice.

II. Genotyping

1. Genomic DNA is extracted from toe tissues of 2-week-old mice, and the genotype of genetically modified mice is identified by PCR and q-PCR methods.

TABLE 1
Information on primer sequences

| Primer | | SEQ |
|---|---|---|
| Name | Primer Sequence | ID NO. |
| Hras12V F | gtagtttaacacattatacact | 1 |
| (700 bp) | | |
| Hras12V R | ctagggctgcaggaattc | 2 |
| qHras12V F | catcaacaacaccaagtcct | 3 |
| (120 bp) | | |
| qHras12V R | gacataaagcctcagtgtgc | 4 |
| IGF-1 F | accagagggaattactatagc | 5 |
| IGF-1 R | tctccagcctccttagatcac | 6 |

Identification on the genotype of Hras12V^{+/+} transgenic mice with hepatocarcinoma: Hras12V transgenes are detected by a common PCR process as positive, and the homozygosis or heterozygosis of Hras12V transgenes is detected by a qPCR process.

Identification on the genotype of IGF-1±transgenic mice: IGF-1 transgenic positive. 10 μL reaction system for a common PCR process: Premix 5 μL, Forward Primer (10 μM) 0.2 μL, Reverse Primer (10 μM) 0.1 μL, DNA 1 μL, ddH₂O 3.6 μL. PCR procedures are: predegeneration at 94° C. for 5 min; degeneration at 94° C. for 1 min, annealing at 60° C. for 30 s, extension at 72° C. for 30 s, 35 cycles; extension at 72° C. for 5 min.

20 μL reaction system for a qPCR process: SYBR Premix 10 μL, ROX 0.4 μL, Forward Primer (10 μM) 0.2 μL, Reverse Primer (10 μM) 0.2 μL, cDNA 1 μL, ddH₂O 8.2 μL. qPCR reaction procedures are: predegeneration at 94° C. for 5 min; degeneration at 94° C. for 1 min, annealing at 60° C. for 20 s, extension at 72° C. for 30 s, 35 cycles; extension at 72° C. for 5 min.

III. Gross Anatomy

Anatomy objects: 6-month-old (M) Hras12V^{+/+} and 6 M/12 M Hras12V^{+/+}/IGF1⁺ transgenic mice, 12 per group.

Anatomy and Sampling:

(1) Blood sampling by eyeball enucleation: Mice are weighed and recorded, then the mice are immobilized with the left hand, making the eyeballs congestion and proptosis. The whiskers of the mice are cut off with a surgical scissor, then the eyeballs are grabbed with a tweezer and removed quickly, making blood to flow from the orbit to the EP tube, which is left at room temperature for 2 h, then centrifuged at 3000 rpm for 30 min, from which serum is sucked up, and stored in a refrigerator at −80° C.

(2) Taking out the liver. Mice are sacrificed by breaking the neck, the anterior abdominal wall between the xiphoid process and the anus is cut-through along the midline of the abdomen, then the lateral abdominal wall is cut-through to the left and right sides along the lowest rib to both sides of the spine, completely exposing organs in the abdomen. Liver is picked, taken a photo, and weighed. The number of liver tumors is recorded and the size of liver tumors is measured with vernier calipers. The liver tumors and the tumor-surrounding tissues are cut into pieces of about 5 mm³ respectively, which are packed in cryogenic tubes, and stored in liquid nitrogen. The remaining tissues are kept in 10% formalin for subsequent pathological examination.

(3) Taking out the spleen (weighing, taking a photo), the stomach and intestine, and the kidney; cutting open the chest, taking out the thymus (taking a photo, and weighing), the heart and the lung, which are kept in 10% formalin for subsequent pathological examination.

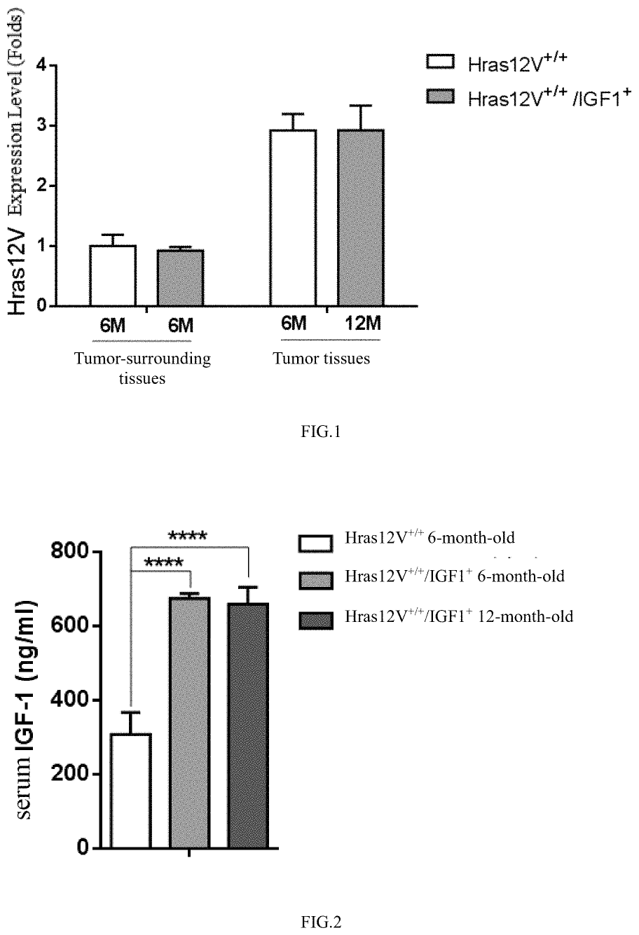

As shown in FIGS. 1-5, wherein FIG. 1 shows that there is no significant difference between mRNA expression levels of Hras12V oncogenes of Hras12V^{+/+} and Hras12V^{+/+}/IGF1⁺ model mice, indicating that there is no significant difference in the inducement intensity of liver tumors; FIG. 2 shows that the IGF-1 level in the serum of Hras12V^{+/+}/IGF1⁺ mice is significantly higher than that in Hras12V^{+/+} mice, indicating that

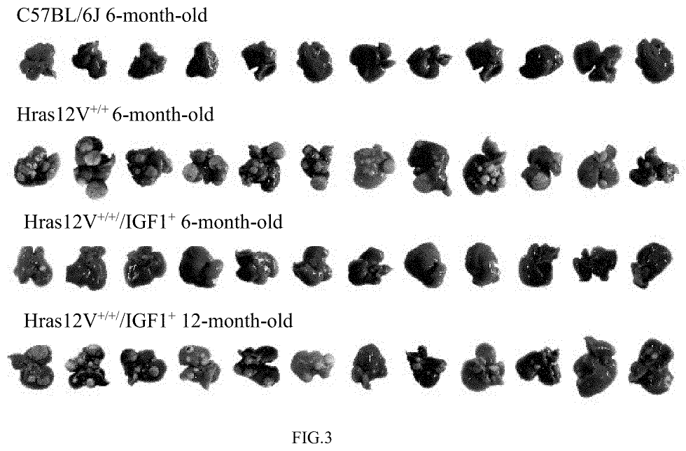

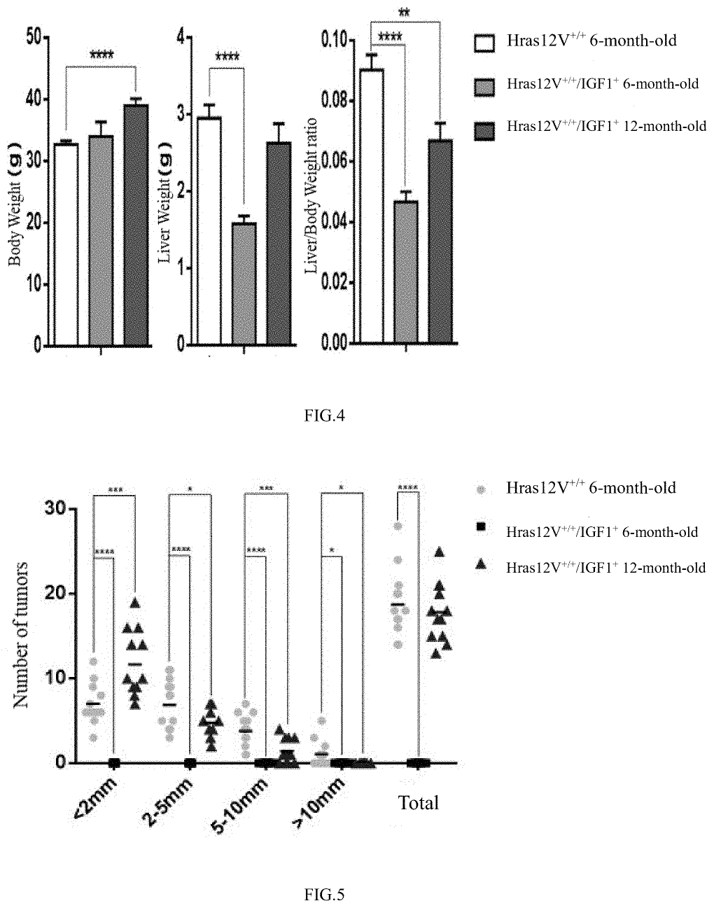

Hras12V transgenic hepatocarcinoma model mice with IGF-1 over-expressed have been successfully established. It is demonstrated by examining the results of the occurrence and development of liver tumors in Hras12V^{+/+} and Hras12V^{+/+}/IGF1⁺ model mice that, the consistency of the data from gross anatomy (FIG. 3), liver/body weight ratio (FIG. 4), and the size and number of liver tumors (FIG. 5) confirmed that over-expression of IGF-1 in hepatocytes significantly inhibits the occurrence and development of liver tumors induced with Ras oncogenes.

### Embodiments 2

Over-Expression of IGF-1 in Hepatocytes Significantly Inhibits the Occurrence and Development of Liver Tumor Induced with Carcinogens

I. Preparation of Model Mice with Induced Hepatocarcinoma

Wild type C57BL/6J male mice, IGF1⁺ transgenic mice, 6˜8-week-old, body weight at 20˜30 g. DEN, CCL4, and alcoholic carcinogens are employed to co-induce liver tumors.

First, DEN is intraperitoneally injected once at 100 mg/kg (formulated in normal saline); 3 days later, starting gavage with CCL4 and olive oil (formulated at a volume ratio of 20:80), 0.05 mL/10 g, 2 times/week; at week 3, intraperitoneal injection of DEN once again (50 mg/kg), at the same time, starting administration of drink water containing 9% of ethanol. From week 4, boostering the dose of CCL4 to 0.08 mL/10 g. During the experiments, mice are fed with pellet feed. The growth situations, changes of mental state and appetite, and changes of physiological indices such as body weight and the like are observed. 20 weeks later, mice are sacrificed, observed, recorded, sampled and detected.

II. Gross Anatomy

The anatomy process is the same as above, eyeballs are picked and blood is sampled, the level of IGF-1 in the serum is detected, the liver is weighed, and the number and size of liver tumors are counted.

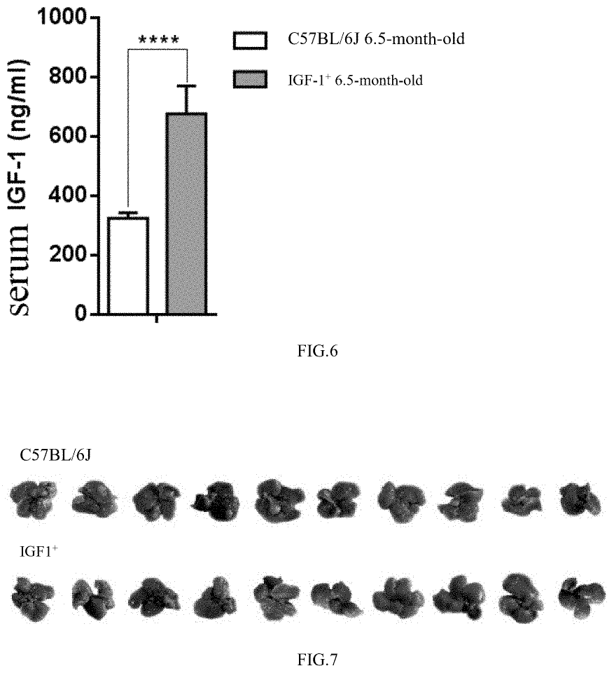

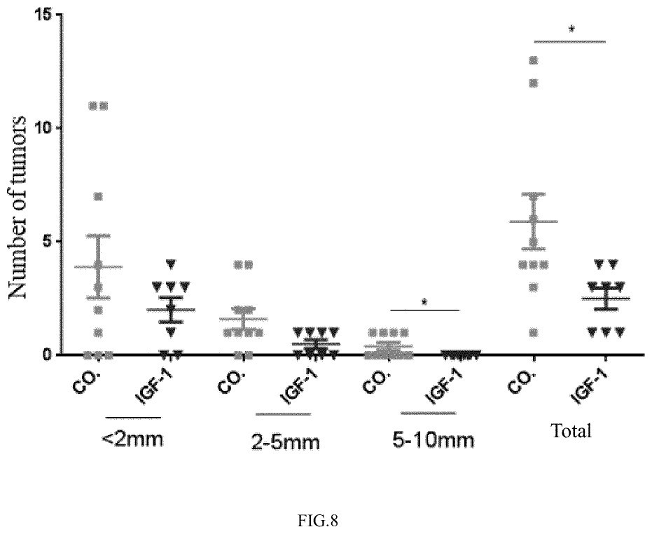

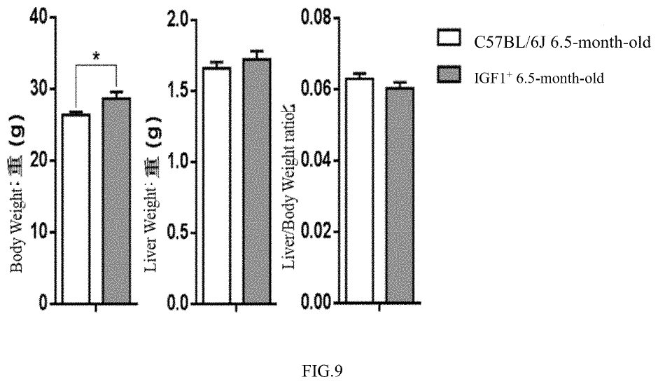

As shown in FIGS. 6-9, wherein FIG. 6 shows that the level of IGF-1 in the serum of IGF1⁺ model mice is significantly higher than that in normal wild type C57BL/6J mice, indicating that the model mice meets the requirements of experimental design. It is demonstrated from the result of examining the occurrence and development of liver tumors in C57BL/6J and IGF1⁺ mice co-induced with DEN, CCL4, and alcoholic carcinogens that, the data from gross anatomy (FIG. 7) and the size and number of liver tumors (FIG. 8) shows consistency, confirming that over-expression of IGF-1 in hepatocytes significantly inhibits the occurrence and development of liver tumors. The result indicates that IGF-1 has an universal effect of antagonizing the occurrence and development of liver tumors. However, as carcinogenic-induced liver tumors are at an early stage, there is no significant difference in liver/body weight ratio (FIG. 9). Additionally, the body weight of IGF1⁺ mice is significantly higher than that of C57BL/6J mice (FIG. 9), indicating the growth-promoting effect of IGF-1.

### Embodiment 3

IGF-1 Induces the Apoptosis of Cancerous Cells Indirectly, and Inhibits the Occurrence and Development of Liver Tumors

I. The role of IGF-1 on the proliferation of hepatocarcinoma cells detected by CCK-8 process

1. Preparation of Cells

Hep3B, HepG2, Hep1-6 cells are resuscitated respectively, cultured in a plate of 10 cm, and passaged serially for 3 times. After the cell growth has stabilized (keeping the cells in a stable state), the cells are digested and harvested to prepare a suspension of single cells.

2. Effect on the Proliferation of Cells

(A) Different concentrations of culture media containing 2-fold IGF-1 concentration are formulated, with a concentration gradient of: 0, 100, 200, 400, 800 ng/mL, each 2 mL.

(B) counting the number of viable cells, adjusting the cell concentration to 5×10⁴/mL, plating on a 96-well plate (0.1 mL/well). Different concentration gradient of working solution is added respectively at 0.1 mL per well, 8 wells for each gradient. The cells are mixed uniformly by blowing with a gun of 200 μL. There are 6 wells without cells used as the blank control, each well is added with 0.2 mL culture medium.

(C) detection of cell activity: a CCK-8 kit is used, the proliferation profile of viable cells is detected at 24 h, and 48 h respectively; 4 parall wells for each time point and each gradient, and 3 parall wells for the blank control.

II. Detection of the Effect on the Proliferation and Apoptosis of Cells by FITC-Annexin V/PI Process

1. Co-Culture of IGF-1 and Cells

The cells are adjusted to a concentration of 2×10⁴/mL, then formulated into a culture solution containing 400 ng/mL IGF-1. A 48-well plate is selected to culture cells, for the blank group, each well is added with 0.5 mL culture medium and 0.5 mL cell suspension and mixed uniformly; for the experimental group, each well is added with 0.5 mL culture medium containing IGF-1 and 0.5 mL cell suspension and mixed uniformly; there are 6 wells for both groups, cells are harvested when cultured to a cell fusion degree of 70%-80%.

2. Harvest of Cells

After digestion of Hep1-6 cells with pancreatic enzymes not containing EDTA, cells are harvested by centrifugation. The cells are washed with pre-cooled PBS for two times. 1×10⁶ cells are harvested and resuspended by adding 100 μL 1×Binding Buffer.

3. Cell Staining

Into the prepared cells are added 5 μL AnnexinV-FITC and 5 μL PI Staining Solution and mixed gently, and reacted for 10 min in dark and at room temperature. 400 μL 1 xBinding Buffer is added and mixed, and the samples are detected with a flow cytometer within 1 h.

III. The Activation of Proliferation and Apoptosis Pathways of Liver Tumors and the Tumor-Surrounding Tissues

1. Extraction of Total Protein

(A) The harvested fresh tissue pieces or tissue pieces cryopreserved in liquid nitrogen are transferred into breaking tubes (each tube is charged with 3-4 breaking beads), into which is added RIPA lysis buffer promptly (tissues taken out from liquid nitrogen need to be operated at low temperature on ice).

(B) Crushing tissues with a biological sample homogenizer, the crused tissue liquid is transferred from the breaking tube to a centrifuge tube of 1.5 mL, left on ice for 1 h.

(C) Low temperature centrifugation at 4° C., at 12000 rpm for 5 min, the supernatant is sucked up and charged into a new centrifuge tube of 1.5 mL, kept at −80° C.

2. Determination of Protein Concentration

(A) Taking BSA mother liquor (5 μg/μL), thawn on ice, mixed with shaking, transient centrifugation for use.

(B) Preparing standard curve samples and BCA working solution according to the instruction of BSA.

(C) Arranging the standard curve and the order of samples to be tested according to the wells of ELISA plate (96 wells, in good order vertically and horizontally). 20 μL standard is taken and added into the corresponding standard curve sample wells of ELISA plate.

(D) Each well with samples to be tested is added 19 μL PBS and 1 μL protein extraction solution, in duplicate for each sample. Each blank control well is added 20 μL PBS. G250 return to room temperature, each well is added with 200 μL, reaction for 8˜15 min.

(E) Measuring protein concentrations following the instruction of ELISA (at a wavelength of 595 nm).

3. Detection of protein expression level by Western blot process

(A) Electrophoresis

Various reagents and instruments required by electrophoresis are checked. The reagents are returned to room temperature. The glass plates for electrophoresis are installed and fixed. Separation gels and stacking gels are formulated, and samples are prepared. The electrophoresis tank is put in proper place, into which is added the electrophoresis buffer. Samples are loaded, the voltage is selected as 80 V when samples are in the stacking gels, and adjusted to 155 V when samples enter the separation gels.

(B) Electrophoresis Transfer

Into a tray is added the electrophoresis buffer. The sponge, filter paper, gel, PVDF membrane, filter paper, and sponge are successively laid on the transfer device from bottom to top, squeezing out the bubbles, and impacted to place on the rack of the transfer, tranferring at 100 V for 2 h.

(C) Immune Response

\textcircled{1} The PVDF membrane at which the target protein is located is cut off according to the position of the protein Marker, blocked with 5% skimmed milk on a shaking bed for 1 h. Excessive skimmed milk is then washed away with 1×TBST, washing 2 times on the shaking bed at a medium rate, 5 min for each time.

\textcircled{2} Primary antibodies are diluted to an appropriate concentration with 1×TBST; the PVDF membrane is placed in an antibody diluent, and incubated at 4° C. on a shaking bed overnight. Washing with 1×TBST on the shaking bed at room temperature for three times, 10 min for each time.

\textcircled{3} Secondary antibodies are diluted with 5% skimmed milk. PVDF membranes are put into the skimmed milk, and then incubated at room temperature for 1 h. They are washed with 1×TBST on the shaking bed at room temperature for 3 times, 10 min for each time, to conduct a chemiluminescence reaction. The developing images are kept in a developing instrument, and the target proteins are labelled.

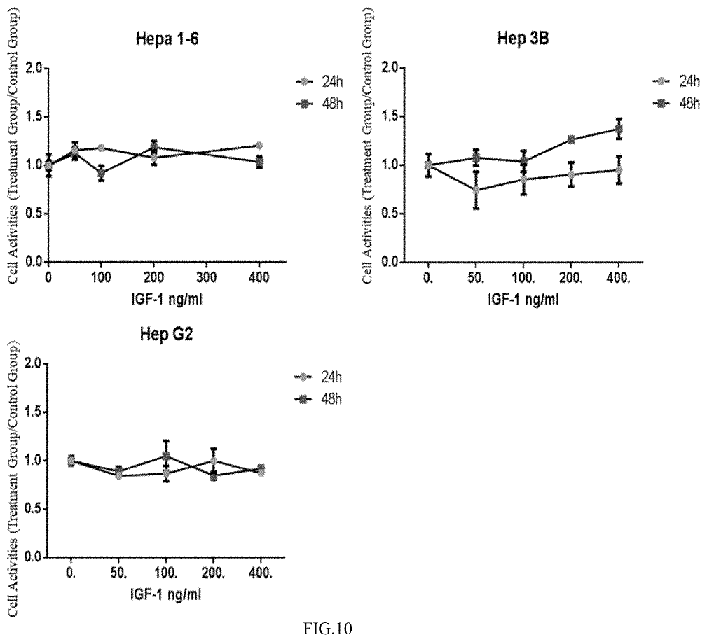

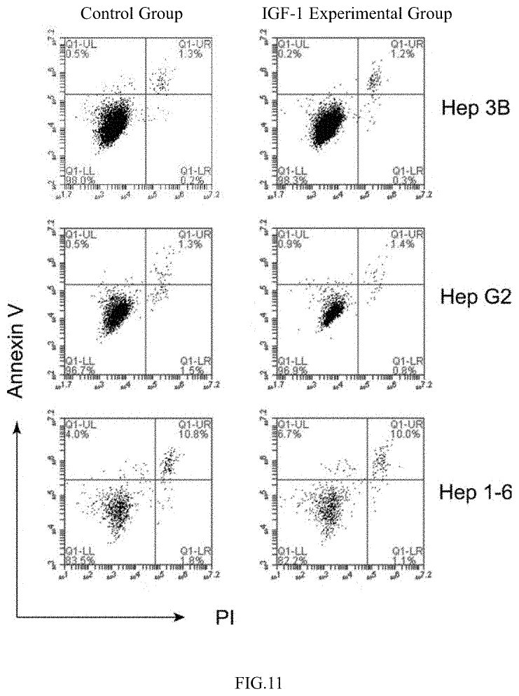

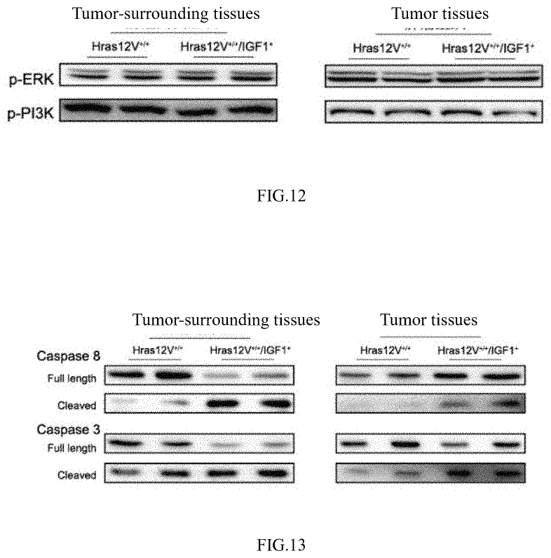

As shown in FIGS. 10-13, 0-400 ng/mL of IGF-1 has no significant effects on the proliferation and apoptosis of Hep3B, HepG2, Hep1-6 hepatocarcinoma cells cultured in vitro (FIGS. 10, 11). It is indicated that IGF-1 itself has no direct effects of inhibiting the proliferation of hepatocarcinoma cells and inducing the apoptosis. It is demonstrated from the result of detecting signaling pathways of liver tumor tissues and the tumor-surrounding tissues in Hras12V^{+/+} mice and Hras12V^{+/+} IGF1⁺ mice that there is no significant difference in the activation levels of MAPK and PI3K/AKT signaling pathways on which IGF-1 directly acts (FIG. 12), indicating that IGF-1 has no significant stimulation effect on the proliferation of liver tumors and the tumor-surrounding tissue cells. However, the apoptosis pathways Caspase 3 and Caspase 8 in liver tumors and the tumor-surrounding tissues of Hras12V^{+/+} IGF1⁺ mice are significantly activated (FIG. 13), indicating that IGF-1 induces the apoptosis of hepatocarcinoma cells indirectly in vivo, further inhibits the occurrence and development of liver tumors.

### Embodiment 4

### High Level of IGF-1 Significantly Promotes the Function of Thymus

Statistics of the weight and index of thymus: 6-month-old (M) Hras12V^{+/+}/IGF1⁺ and Hras12V^{+/+} transgenic mice, 12 per group are selected as the subjects of the study to count the weight of thymus and calculate the thymus index.

Statistics of the proportion of thymus T cells: To avoid the effect of liver tumors on the varieties and quantity of mice lymphocytes, 3-month-old (M) Hras12V^{+/+}/IGF1⁺ and Hras12V^{+/+} transgenic mice, 12 per group are selected as the subjects of the study to count the proportion of thymus T lymphocytes.

I. Counting on Thymocytes

1. Mice are sacrificed by breaking the neck, the thymus is taken out, and fats and connective tissues on the thymus are completely removed.

2. The thymus is cut into pieces with a scissor in 5 mL of RPMI-1640 culture medium, then blown with a gun regularly and slowly for 5 min (releasing thymocytes as many as possible), and the sample solution is transferred into a 15 mL centrifuge tube.

3. Into the culture medium containing thymus debris is added 5 mL containing 1.25% (w/v) of collagenase and 1% (w/v) of DNA enzyme, incubated at 37° C. for 30 min, and the thymus is blown mildly every 15 min.

4. The supernatant is harvested and placed on ice. The sedimentated thymus debris is re-digested (the same as step 3) repeatedly for 2 times until the thymus is digested completely.

5. All the harvested supernatants are combined and centrifuged for 5 min under 472 g centrifugal force, discarding the supernatant. The cells are resuspended in 1 mL cold PBS (containing 5 mM EDTA, 0.1% FBS), and filtered over a nylon screen of 200 meshes, counting by microscopy.

II. Proportion of Thymus T Lymphocytes

1. Mice are sacrificed by breaking the neck, the thymus is taken out, and fats and connective tissues on the thymus are completely removed, and placed in a sterile culture dish.

2. The thymus is cut into pieces with a scissor in 2 mL of PBS, then transferred into a homogenizer to be gound until there is no larger debris.

3. The homogenate is transferred into a 15 mL centrifuge tube, into which is added 3 mL PBS to wash thymocytes with shaking.

4. Centrifugation at 1000 rpm for 5 min, discarding the supernatant, into which is added 3 mL PBS and blown to mix uniformly, filtering over a screen of 200 meshes.

5. Diluting the cell suspension by a factor of 100 and counting, detecting the cell state under a microscope, and calculating the total number of cells.

6. Resuspending the cells in 100 μL PBS, and adjusting the cell concentration to 5×10⁵˜1×10⁶/mL.

7. Adding CD4, CD8a and CD3e antibodies, each 0.5 μL, vortex shaking, then incubation at 4° C. and in dark for 30 min.

8. Adding 1 mL PBS to wash the cells, centrifugation at 160 g for 5 min, discarding the supernatant.

9. Adding 200 μL PBS and shaking, then detection on the instrusment.

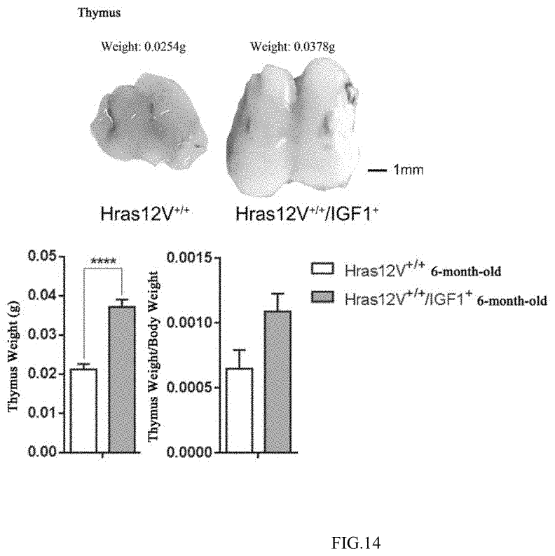

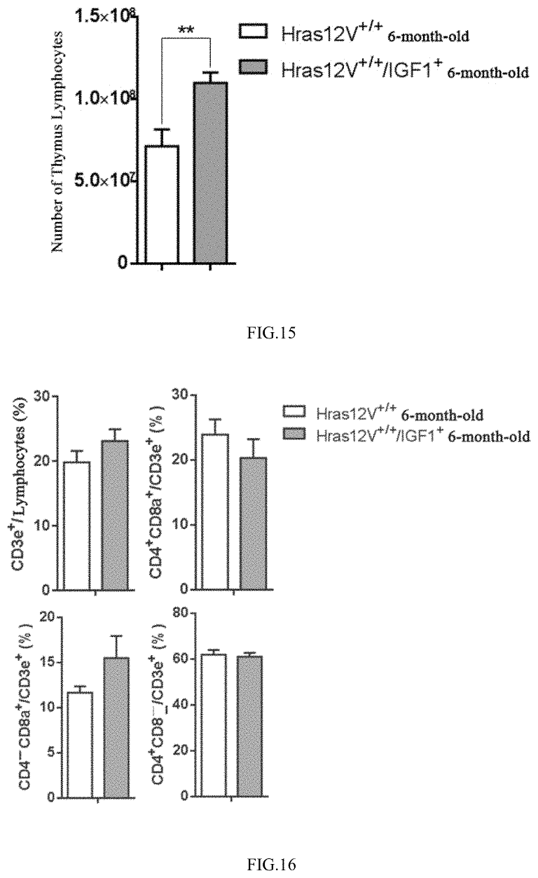

As shown in FIGS. 14-16, the weight, index, and the number of cells of the thymus of 6-month-old Hras12V^{+/+}/IGF1⁺ mice are all significantly higher than those of Hras12V^{+/+} mice (FIGS. 14 and 15, wherein the left drawing in FIG. 14 is the comparative entity photo), indicating that high level of IGF-1 effectively promotes the function of thymus, and delays the degeneration of thymus. However, there are no significant changes in the proportion of T lymphocyte subtypes in the thymus of Hras12V^{+/+}/IGF1⁺ and Hras12V^{+/+} mice (FIG. 16), indicating that high level of IGF-1 does not causes the dysfunction of thymus.

### Embodiment 5

High Level of IGF-1 Significantly Promotes the Function of Spleen T Lymphocytes

### Technical Solution

1. Statistics of the weight and index of spleen: 6 M Hras12V^{+/+} and 6 M Hras12V^{+/+}/IGF1⁺ transgenic mice, 12 per group are selected as the subjects of the study to count the weight of spleen and calculate the spleen index.

2. Detection of the number of spleen lymphocytes

(A) Adding 4 mL mice lymphocyte separation medium (return to room temperature and shake well before use) into a glass grinder to grind the tissues.

(B) Immediately transferring a separation medium of spleen cells into a 15 mL centrifuge tube, which is covered with 1000 μL of RPMI1640 culture medium (keeping a clear boundary of liquid level).

(C) At room temperature, centrifugation at 800 g for 30 min. After centrifugation, cells are layered.

(D) Sucking out the layer of lymphocytes, then adding 10 mL RPMI1640 culture medium, and washing inversely. At room temperature, centrifugation at 250 g for 10 min to collect the cells.

The supernatant is decanted, and cells are resuspended with serum-free medium or other culture solutions and counted.

3. Detection of Spleen T Lymphocytes

(A) Resuspending spleen single cells in 100 μL PBS, and adjusting the cell concentration to 5×10⁵˜1×10⁶/mL.

(B) Adding CD4, CD8a and CD3e antibodies, each 0.5 μL, vortex shaking, then incubation at 4° C. and in dark for 30 min.

(C) Adding 1 mL PBS to wash the cells, centrifugation at 160 g for 5 min, discarding the supernatant.

(D) Adding 200 μL PBS and shaking, then detection on the instrusment.

4. Detection on the Activity of Spleen Cells

(A) The lymphocyte resuspension is formulated as shown in Table 2.

TABLE 2
Formulation of lymphocyte resuspension

| | | | | | Dose of 5 mL culture | |
|---|---|---|---|---|---|---|
| | | | | | medium (2 folds of | |
| | Concentrated | | Required | | medicament | |
| Name | Stock Solution | | Concentration | | concentration) | |
| PMA | 50 | μg/mL | 50 | ng/mL | 10 | μL |
| Ionomycin | 1 | mg/mL | 1 | μg/mL | 10 | μL |
| BFA | 2.5 | mg/mL | 10 | μg/mL | 4 | μL |

| | | | |
|---|---|---|---|
| Culture medium (10% FBS) | Up to | 5 | mL |

(B) After resuspension of lymphocytes, cells are counted, and the concentration is adjusted to 2×10⁶/mL. Each well is added with 0.5 mL (1×10⁶ cells), plated in a 48-well plate. Each sample is added into 5 wells in parallel (separate staining of IFN-γ and IL-2), then the formulated culture medium is added at 0.5 mL/well, placed in an incubator at 37° C. and stimulated for 4 h.

(C) Harvest of cells: the cells are harvested into a 1.5 mL EP tube, centrifuged at 1500 rpm for 5 min (kept at 4° C.), and the cell precipitates are collected.

(D) Adding 1 mL PBS to wash the cells by vortex shaking, centrifugation at 1500 rpm for 5 min, discarding the supernatant.

(E) The remaining about 100 μL are resuspended with shaking, 100 μL is used to lable CD3e, CD8, CD4, which are incubated at 4° C. and in dark for 30 min.

(F) PBS washing: the contents of each tube are washed by adding 1 mL PBS, and centrifuged at 1500 rpm for 5 min, discarding the supernatant, with the remainder of about 50 μL PBS, and the cells are dispersed with vortex.

(G) Fixation and membrane rupture: Each tube is added with 250 μL membrane rupturing liquid, incubated at 4° C. and in dark for 20 min, and shaked every 10 min.

(H) Washing: Washing cells with 1 mL Perm/Wash buffer, washing with (10×Perm/Wash buffer diluted with ddH₂O) once, centrifugation at 250 g for 5 min, discarding the supernatant.

(I) Into the remaining 50 μL reaction system is added 0.5 μL antibodies labelling IFN-γ [symbol] IL-2 intracellular cytokines, and incubated at 4° C. and in dark for 30 min.

(J) Washing: Each tube is added with 1 mL Perm/Wash buffer for washing once, centrifugation at 250 g for 5 min, discarding the supernatant. 200 μL cell resuspension in 1×BD Perm/Wash is loaded on the instrument.

5. ConA stimulates the proliferation of mice spleen lymphocytes

(A) ConA concentrated stock solution is diluted with RPMI-1640 culture medium to a working solution at a concentration of 5 μg/mL.

(B) Counting on lymphocytes: spleen lymphocytes are resuspended with RPMI 1640 culture medium (FBS-free), and counted by a blood counting chamber

(C) CFSE-labelled spleen cells: A portion of cells are used as the negative control group without staining, and into other cells is added with CFSE staining fluid (the final concentration of the staining fluid is 2.5 μM), incubated at room temperature and in dark for 9 min, added with 10-fold volume of culture medium containing 10% of FBS, mixed well by blowing, incubated at room temperature for 9 min, and added with 10-fold volume of complete culture medium, left at room temperature for 2 min to stop the reaction.

(D) Washing: Discarding the supernatant by centrifugation, tapping the bottom of the centrifuge tube, and dispensing the cells. 3 mL culture medium containing 10% of FBS is added and mixed well by blowing. Centrifugation at 1000 rpm for 5 min, discarding the supernatant, which are repeated for 2 times.

(E) Plating: The concentration of lymphocytes is adjusted to 3×10⁶/mL. 100 μL cells are added into a 96-well plate, with 3 wells in parallel for each group. A system is formulated in a 15 mL centrifuge tube, plated in a 96-well type U plate, and then 100 μL of ConA working solution is added.

(F) Incubation: Lymphocytes are incubated in an incubator at 37° C. for 3 days, then detected by a flow cytometer.

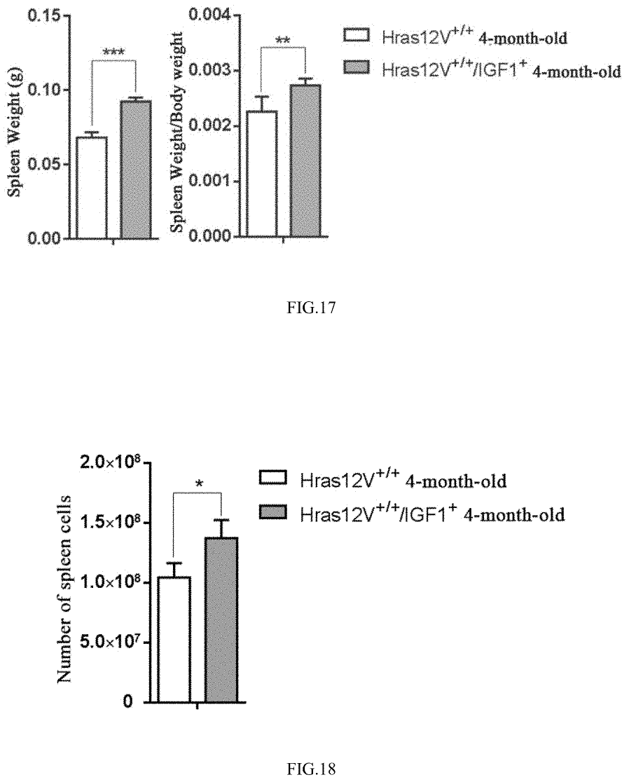

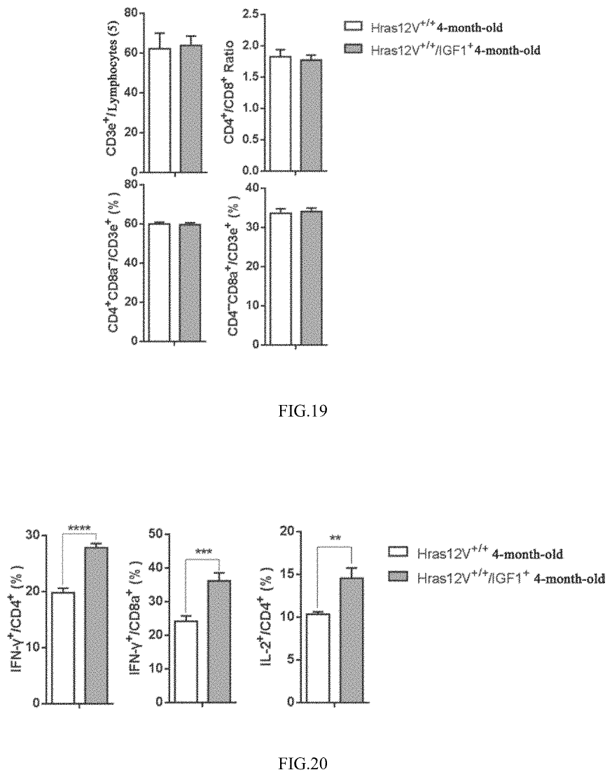

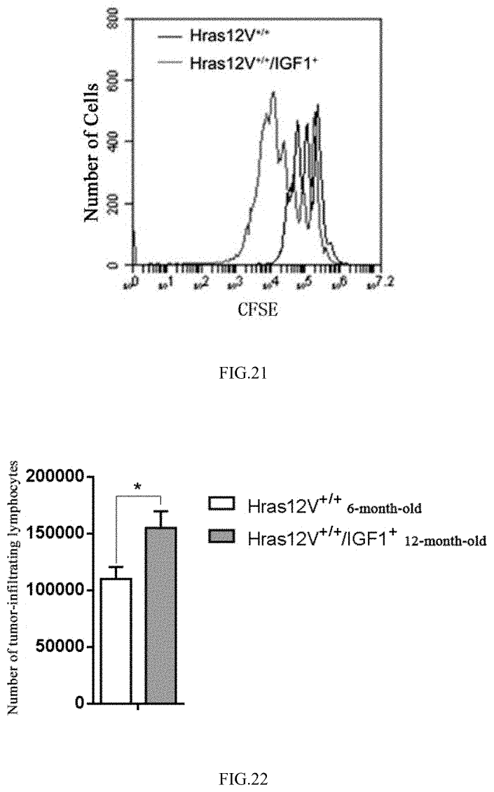

As shown in FIGS. 17-21, the weight, index, and the number of cells of the spleen of 4-month-old Hras12V^{+/+}/IGF1⁺ mice are all significantly higher than those of Hras12V^{+/+} mice (FIGS. 17 and 18), indicating that high level of IGF-1 effectively promote the function of spleen. However, there are no significant changes in the proportion of T lymphocyte subtypes in the spleen of Hras12V^{+/+}/IGF1⁺ and Hras12V^{+/+} mice (FIG. 19), indicating that high level of IGF-1 does not causes the dysfunction of spleen. It is further demonstrated from the detection results on the activity of spleen cells that, after stimulation of PMA and calcium ions, the secretion volumes of IFN-γ and IL-2 of CD4 T lymphocytes, as well as the secretion volume of IFN-γ of CD8 T lymphocytes in the spleen of Hras12V^{+/+}/IGF1⁺ mice are significantly higher than those in Hras12V^{+/+} mice (FIG. 20). Additionally, it is demonstrated from the experimental results about the stimulation of ConA on the proliferation of spleen lymphocytes in mice that, the proliferation capacity of spleen T lymphocytes in Hras12V^{+/+}/IGF1⁺ mice is significantly higher than that in Hras12V^{+/+} mice (FIG. 21). These results show that high level of IGF-1 promotes the activity and proliferation capacity of T lymphocytes in peripheral lymphoid organs.

### Embodiment 6

### High Level of IGF-1 Significantly Enhances the Number and Activity of Tumor Infiltrating T Lymphocytes

I. Total Number of Lymphocytes

1. Separation of Tumor Infiltrating Lymphocytes with Percoll Fluid

(1) Formulation of NaCl Solution and Gradient Percoll Fluid

A. 8.5 g NaCl solid is weighed and dissolved with a small amount of distilled water in a clean beaker. The liquid is transferred into a 100 mL volumetric flask, metered, and sterilized, being ready for use. (8.5% NaCl=1.5 M NaCl)

B. Preparation of 0.85% NaCl: 5 mL 8.5% NaCl+45 mL sterile water in a 50 mL centrifuge tube, being ready for use.

100% Percoll=9 mL Percoll Fluid+1 mL of 8.5% NaCl

70% percoll=2 mL 100% Percoll+0.773 mL of 0.85% NaCl

50% percoll=2 mL 100% Percoll+1.795 mL of 0.85% NaCl

30% percoll=2 mL 100% Percoll+4.167 mL of 0.85% NaCl

(2) Preparation of Samples

A. Tumor tissues are stripped aseptically and weighed, with the tumor weight in a range of 0.35±0.05 g.

B. 2 mL PBS is added into a homogenizer, the weighed tissues are ground in the homogenizer and filtered over a filter screen of 200 meshes into a 15 mL centrifuge tube, into which is added with 1×PBS to 5 mL and mixed well by blowing, inverted upside down, and centrifuged at 1500 rpm for 5 min.

C. Discarding the supernatant, and resuspending with 2 mL serum-free RPMI1640 culture medium.

(3) Operation Steps

A. Preparation of Percoll layers with discontinuous density gradient: firstly, the walls of a 15 mL centrifuge tube are wetted with fetal bovine serum, with the extra serum discarded.

B. Adding Percoll at concentrations of 70%, 50%, 30% successively, each 2 mL.

C. Adding the cell suspension slowly onto the liquid level of the test tube containing Percoll fluid.

(4) Centrifugation: at 2600 rpm, centrifugation for 30 min (centrifugation at room temperature).

(5) Sampling: Sucking out the white mononuclear lymphocyte layer carefully (located at the third layer).

(6) Washing cells: adding 1×PBS to 5 mL, centrifugation at 1500 rpm for 5 min, discarding the supernatant. Further adding 1×PBS 3 mL, centrifugation at 1500 rpm for 5 min, discarding the supernatant, adding 300 μL RPMI1640 culture medium for resuspension, filtering over a screen of 200 meshes, and ready for use.

(7) Determination of cell purity (flow cytometry): Cells are diluted by 10 times, filtered over a screen of 200 meshes and determined on an instrument.

II. Detection on Proportion and Activity of T Lymphocytes (the Same as Spleen Lymphocytes)

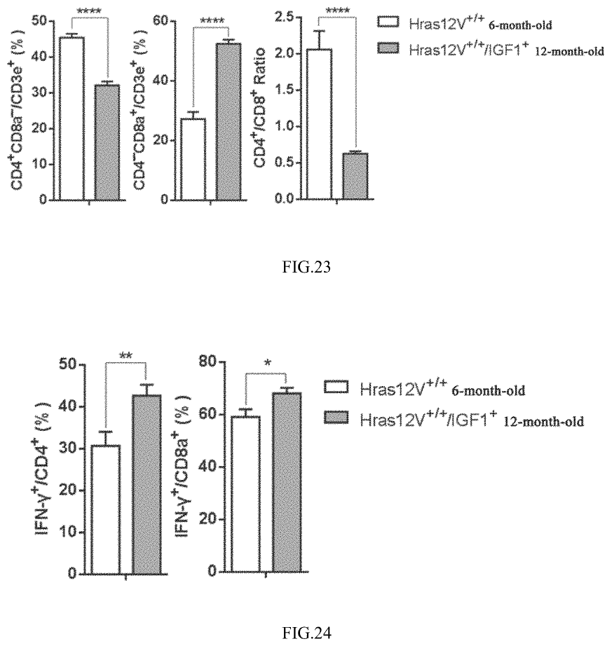

As shown in FIGS. 22-24, it is demonstrated from the analysis results on the infiltrating lymphocytes of liver tumor tissues which are at the same development stage and similar in size that, the number of tumor infiltrating lymphocytes, the proportion of CD8/CD4 T lymphocytes and the activity of T lymphocytes in Hras12V^{+/+}/IGF1⁺ mice are all significantly higher than those in Hras12V^{+/+} mice (FIGS. 22-24). These results show that high level of IGF-1 can effectively enhance the number and activity of tumor infiltrating T lymphocytes, and inhibit the tumor development.

### Embodiment 7

Intraperitoneal Injection of IGF-1 can Effectively Inhibit the Occurrence and Development of Liver Tumor Induced with Ras Oncogenes

3-month-old SPF-grade Hras12V^{+/+} transgenic hepatocarcinoma male mice (24) are randomly devided into two groups, 12 per group, experimental group (IGF-1) and control group (Co.) respectively. For the experimental group, intraperitoneal injection of 600 μL IGF-1 (312.5 mg/kg/day) is conducted every morning and evening. While for the control group, synchronous intraperitoneal injection of 600 μL mannitol solvent at equal concentration (300 mg/L) is conducted. 6 months after the treatment, experimental mice are subjected to anatomy, observing the effect of exogenous IGF-1 on the occurrence and development of liver tumors induced with Ras oncogenes.

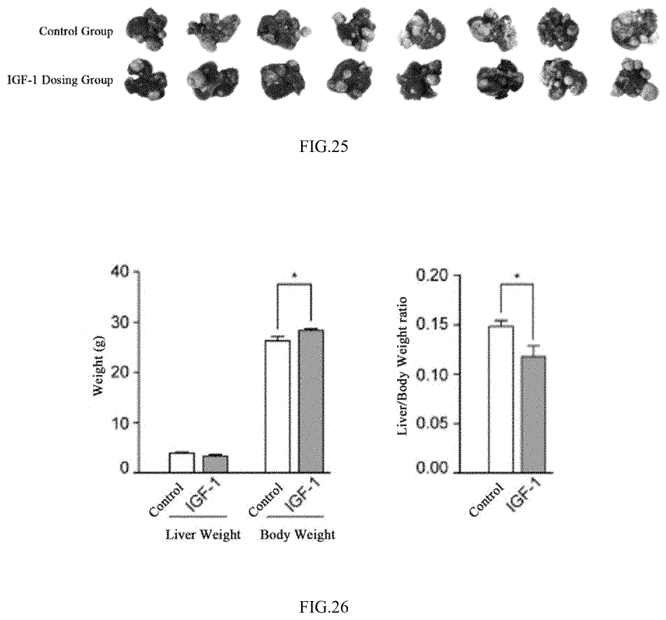

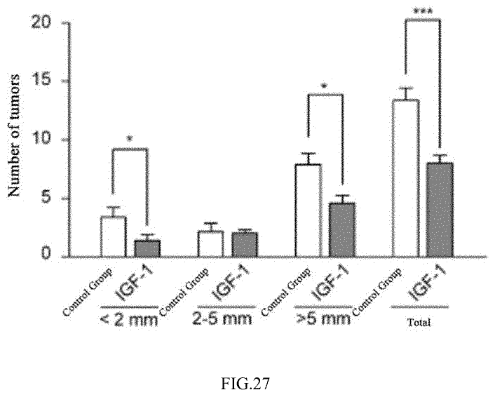

As shown in FIGS. 25˜27, exogenously increasing the level of IGF-1 in the body can effectively inhibit the occurrence and development of liver tumors induced with Ras oncogenes (FIGS. 25˜27), indicating that exogenously increasing the level of IGF-1 in the body has a significant inhibition on the occurrence and development of liver tumors.

In conclusion, the present invention provides a use of IGF-1 in the preparation of medicaments for preventing and/or treating liver tumors. According to the experimental results on mice, when the level of IGF-1 in the body is 2˜3 times of the normal concentration of IGF-1 in the serums, there will be significant inhibition on the occurrence and development of liver tumors induced with Ras oncogenes. Moreover, long-term over-expression of IGF-1 has no significant promotion on the incidence of tumors in all organs and tissues of mice, indicating the safety of high IGF-1 level in the body; and IGF-1 itself has no direct inhibition on liver tumor cells, and enhancing the activity of T cells is an important way for IGF-1 to indirectly inhibit the occurrence and development of liver tumors.

The foregoing is only the preferable embodiment of the invention, it should be noted to persons with ordinary skills in the art that several improvements and modifications can be made to the invention without deviating from the principle of the invention, which are also considered to be in the protection scope of the invention.

What is claimed is:

1. A method for preventing and/or treating liver tumors with IGF-1, comprising improving the level of IGF-1 in the body.

2. The method of claim 1, wherein the level of IGF-1 in the body is improved with exogenous IGF-1.

3. The method of claim 2, wherein the exogenous IGF-1 is ingested by means of injection, gavage, swallowing or gene transfection techniques.

4. The method of claim 1, wherein the prevention and/or treatment of liver tumors is achieved by promoting the function of thymus with IGF-1.

5. The method of claim 2, wherein the prevention and/or treatment of liver tumors is achieved by promoting the function of thymus with IGF-1.

6. The method of claim 3, wherein the prevention and/or treatment of liver tumors is achieved by promoting the function of thymus with IGF-1.

7. The method of claim 1, wherein the prevention and/or treatment of liver tumors is achieved by promoting the function of spleen T lymphocytes with IGF-1.

8. The method of claim 2, wherein the prevention and/or treatment of liver tumors is achieved by promoting the function of spleen T lymphocytes with IGF-1.

9. The method of claim 3, wherein the prevention and/or treatment of liver tumors is achieved by promoting the function of spleen T lymphocytes with IGF-1.

10. A medicament for preventing and/or treating liver tumors, comprising IGF-1 and excipients.


SEQUENCE LISTING (6 sequences)

SEQ ID NO: 1 - DNA, 22 bp, artificial sequence
  Hras12V F
gtagtttaacacattatacact

SEQ ID NO: 2 - DNA, 18 bp, artificial sequence
  Hras12V R
ctagggctgcaggaattc

SEQ ID NO: 3 - DNA, 20 bp, artificial sequence
  qHras12V F
catcaacaacaccaagtcct

SEQ ID NO: 4 - DNA, 20 bp, artificial sequence
  qHras12V R
gacataaagcctcagtgtgc

SEQ ID NO: 5 - DNA, 21 bp, artificial sequence
  IGF-1 F
accagagggaattactatagc

SEQ ID NO: 6 - DNA, 21 bp, artificial sequence
  IGF-1 R
tctccagcctccttagatcac